(19) United States
(12) Patent Application Publication
(10) Pub. No.: US 2022/0214348 A1
(43) Pub. Date: Jul. 7, 2022

(54) KIT FOR DETECTING MASTITIS IN DAIRY COWS AND APPLICATION METHOD THEREOF

(71) Applicant: Biology Institute, Hebei Academy Of Sciences, Shijiazhuang (CN)

(72) Inventors: Meng Wu, Shijiazhuang (CN); Zhijing Wei, Shijiazhuang (CN); Chunsheng Li, Shijiazhuang (CN); Yingzhu Chen, Shijiazhuang (CN)

(21) Appl. No.: 17/265,903

(22) PCT Filed: Sep. 1, 2020
(86) PCT No.: PCT/CN2020/112790

§ 371 Date: Feb. 4, 2021

(30) Foreign Application Priority Data
Sep. 26, 2019 (CN) 201910916156.7

(51) Int. Cl. G01N 33/58; G01N 33/543

(52) U.S. Cl. CPC G01N 33/582; G01N 2800/365; G01N 33/54388

(57) ABSTRACT

The present invention relates to a fluorescence immunochromatographic assay kit and an application method thereof, belonging to the technical field of in vitro diagnostic products for animal diseases. The kit comprises a plastic case, a test reagent card and a sample diluent. The case comprises a bottom case and an upper cover, wherein a test strip slot is formed in the bottom case, and a scan window and a sample loading hole are arranged on the upper cover; wherein the test reagent card consists of a sample pad, a binding pad, a nitrocellulose membrane and an absorbent paper which are sequentially adhered on a bottom board; the position of the scan window is matched with the position of the nitrocellulose membrane, and the position of the sample loading hole is matched with the position of the sample pad.

### FIELD OF THE INVENTION

The present invention relates to a fluorescence immunochromatographic assay kit and an application method thereof, in particular to a fluorescence immunochromatographic assay kit for detecting mastitis in dairy cows and an application method thereof, belonging to the technical field of in-vitro diagnostic products for animal diseases.

### BACKGROUND OF THE INVENTION

Mastitis is a common disease in the process of raising dairy cows. Once dairy cows are attacked by the disease, the quality of milk will be seriously affected. If it is impossible to accurately and timely detect whether dairy cows are suffering from mastitis, it will cause huge economic losses to dairy stations and dairy enterprises. At present, mastitis is mainly screened by somatic cell counting (SCC), but the sensitivity and accuracy of SCC are limited and susceptible to other factors, so that the inflammatory state cannot be reflected accurately. At present, there are few researches on the diagnosis of mastitis in dairy cows in China, so it is urgent to develop assay products associated with mastitis in dairy cows, improve the detection level and provide people with safe high-quality dairy products.

Acute phase proteins (APPs) are a family of recognized protein indicators of inflammation, trauma and other pathological conditions, including haptoglobin, serum amyloid A, C-reactive protein and so on. Among them, the content of bovine serum amyloid A can increase rapidly under the stimulation of acute inflammation or tissue damage. During inflammation, the main function of bovine serum amyloid A is to remove damaged tissues, induce adhesion and chemotaxis of macrophages and lymphocytes, and enhance their bactericidal and phagocytic functions. The secretion of bovine serum amyloid A gradually decreases to the normal level after the inflammation is relieved. It has been reported that the content of serum amyloid A in milk is positively correlated with the incidence of mastitis in dairy cows. According to some literatures, the detection results of bovine serum amyloid A are more sensitive than those of SCC, and less susceptible to interference. Therefore, quantitative detection of bovine serum amyloid A is a more valuable and promising diagnostic method for mastitis in dairy cows.

Immunochromatography is a rapid immunoassay technique using a nitrocellulose membrane as a solid carrier. A sample to be tested flows on the nitrocellulose membrane by capillary action, the sample to be tested, if containing a target antigen (or antibody), will bind to a tracer labelled with the antibody (or antigen) to form a complex which will be captured by the antibody (or antigen) in a specific area of the nitrocellulose membrane. The content of a target antigen (or antibody) in the sample to be tested can be obtained by detecting the tracer in the specific area.

Time-resolved fluorescence immunoassay is a non-radioimmunoassay technique using lanthanides as markers. Lanthanides have the advantages of long fluorescence half-life, large stokes shift, wide excitation spectra and narrow emission spectra, as a result, the technique has high detection sensitivity.

Fluorescence immunochromatography using time-resolved fluorescent microspheres has the advantages of safe and fast operation, suitability for single test, quantitative detection, high sensitivity, good specificity and low cost.

The existing detection products of milk serum amyloid A are only ELISA detection kit, which takes a long time and is not suitable for single sample detection.

### SUMMARY OF THE INVENTION

In order to overcome the disadvantages in the prior art, the present invention provides an immunochromatographic quantitative kit for determining milk serum amyloid A based on fluorescence immunochromatography. The kit can judge whether a dairy cow suffers from mastitis or the severity of mastitis by detecting the content of serum amyloid A in milk, and has the characteristics of fast operation, accuracy and low cost.

The technical problem in the present invention is solved by the following technical solution.

A fluorescence immunochromatographic assay kit for detecting mastitis in dairy cows, comprising a plastic case, a test reagent card and a sample diluent, wherein the case comprises a bottom case and an upper cover, wherein a test strip slot is formed in the bottom case, and a scan window and a sample loading hole are arranged on the upper cover; wherein the test reagent card consists of a sample pad, a binding pad, a nitrocellulose membrane and an absorbent paper which are sequentially adhered on a bottom board; the position of the scan window is matched with the position of the nitrocellulose membrane, and the position of the sample loading hole is matched with the position of the sample pad.

According to the fluorescence immunochromatographic assay kit for detecting mastitis in dairy cows, both the sample pad and the binding pad are glass cellulose membranes, and the bottom board is a PVC bottom board.

According to the fluorescence immunochromatographic assay kit for detecting mastitis in dairy cows, the sample pad is pretreated with a sample pad pretreatment buffer which is prepared by dissolving a sample pad buffer, a sample pad protein protectant and a sample pad surfactant in water; wherein, the sample pad buffer is selected from any one of PBS buffer, Tris-HCl buffer, borate buffer and citric acid-sodium citrate buffer, with a concentration of 5-100 mM; the sample pad protein protectant is selected from any one or more of BSA, gelatin from cold water fish skin, casein, casein sodium salt and bovine serum, with a ratio of dosage (g) to total volume (L) of sample pad pretreatment buffer of 0.5-20 g:1; the sample pad surfactant is selected from any one of Tween-20, Tween-80, TritonX-100 and TritonX-305, with a ratio of dosage (g) to total volume (L) of sample pad pretreatment buffer of 2-20 g:1; and pH value of the sample pad pretreatment buffer is adjusted by using a pH regulator commonly used in the prior art, with a range of 7.0-8.0.

According to the fluorescence immunochromatographic assay kit for detecting mastitis in dairy cows, the binding pad contains a complex of fluorescent microsphere-labelled chicken IgY and a fluorescent microsphere-labelled monoclonal antibody against bovine serum amyloid A; wherein the binding pad is pretreated by using a binding pad pretreatment buffer which is prepared by dissolving a binding pad protein protectant, a binding pad reaction enhancer and a binding pad surfactant in water; wherein, the binding pad protein protectant is selected from any one or more of bovine serum albumin (BSA), gelatin from cold water fish skin, casein, casein sodium salt, bovine serum, sucrose and trehalose, with a ratio of dosage (g) to total volume (L) of binding pad pretreatment buffer of 0.5-50:1; the binding pad reaction enhancer is selected from any one of PEG6000, PEG8000, PEG20000, PVP K30 and PVP K40, with a ratio of dosage (g) to total volume (L) of binding pad pretreatment buffer of 0.1-10:1; and the binding pad surfactant is selected from any one of Tween-20, Tween-80, TritonX-100 and TritonX-305, with a ratio of dosage (g) to total volume (L) of binding pad pretreatment buffer of 0.5-10:1.

According to the fluorescence immunochromatographic assay kit for detecting mastitis in dairy cows, the nitrocellulose membrane is coated with a test line of a monoclonal antibody against bovine serum amyloid A and a quality control line of a rabbit anti-chicken IgY antibody, wherein the test line is close to the binding pad and the quality control line is close to the absorbent paper.

According to the fluorescence immunochromatographic assay kit for detecting mastitis in dairy cows, the binding pad and the sample pad are pretreated in the following steps: soaking the binding pad or the sample pad in the binding pad pretreatment buffer or the sample pad pretreatment buffer respectively for 0.5-2 h, then taking out and drying the binding pad or the sample pad at 36-38° C.

The fluorescence immunochromatographic assay kit for detecting mastitis in dairy cows is based on the principle of quantitatively detecting the content of serum amyloid A in a milk sample by a double antibody sandwich method which comprises the following steps: dripping the sample into the sample loading hole to allow the sample to flow into the binding pad by chromatography, wherein the sample, if containing bovine serum amyloid A, binds to the fluorescent labelled monoclonal antibody against bovine serum amyloid A on the binding pad to form an immune complex, the complex and the fluorescent labelled chicken IgY continue to move to the nitrocellulose membrane where the complex specifically binds to a T line coated monoclonal antibody against bovine serum amyloid A, finally forming a double antibody sandwich complex, and the fluorescent labelled chicken IgY binds to a C line coated rabbit anti-chicken IgY antibody; measuring and analyzing fluorescence values of the T line and the C line by using a quantitative fluorescence analyzer; plotting a calibration curve based on the relationship between the fluorescence ratio of T/C measured by the kit and the concentration of a calibrator, substituting the measured fluorescence ratio of T/C into the calibration curve, and calculating the content of bovine serum amyloid A in the sample.

An application method of the fluorescence immunochromatographic assay kit for detecting mastitis of dairy cows is as follows: putting the milk sample into a centrifuge tube filled with a sample diluent (0.01 M phosphate buffer) immediately after sample collection, with a volume ratio of the milk to the sample diluent of 1:5, then shaking the centrifuge tube to mix the resulting mixture well; during assay, dripping 100 μL of diluted sample into the sample loading hole, and allowing to stand horizontally for reaction at room temperature for 5 min; after the reaction, inserting the test reagent card into the quantitative fluorescence analyzer, reading the fluorescence signal values of C and T lines, calculating the corresponding T/C value, substituting the calculated T/C value into the calibration curve, and calculating the concentration of bovine serum amyloid A in the sample.

The kit for detecting mastitis in dairy cows developed in the present invention detects serum amyloid A in milk by fluorescence immunochromatography for the first time, and has the advantages of fast and simple operation, accuracy and low cost.

### BRIEF DESCRIPTION OF THE FIGURES

FIG. 1 is a section view of a fluorescence immunochromatographic kit for bovine serum amyloid A of the present invention.

FIG. 2 is a section view of a fluorescence immunochromatographic test strip for bovine serum amyloid A of the present invention.

FIG. 3 shows a calibration curve of a fluorescence immunochromatographic kit for bovine serum amyloid A of the present invention.

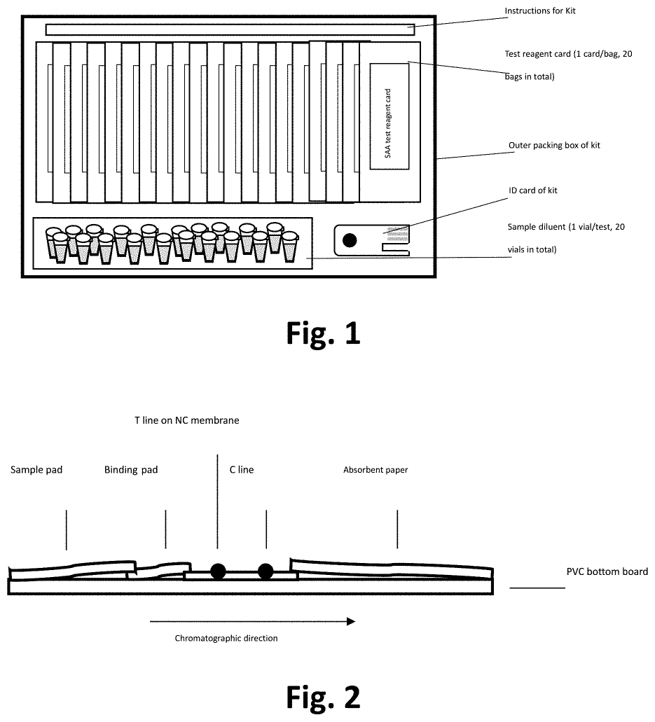

### DESCRIPTION OF THE INVENTION

### Embodiment 1: Preparation of a Fluorescence Immunochromatographic Assay Kit for Bovine Serum Amyloid A

1) Coating:

Adhering a nitrocellulose membrane to the middle of a bottom board, diluting a monoclonal antibody against bovine serum amyloid A (purchased from Medix Biochemica) to 2 mg/ml with a 0.01M PB buffer as a T line coating solution, and diluting a rabbit anti-chicken IgY antibody (purchased from Nanjing Hanrui Baike Biotechnology Co., Ltd.) to 2 mg/ml with a 0.01M PB buffer as a C line coating solution; coating the T line coating solution and the C line coating solution on the nitrocellulose membrane by using an XYZ platform dispenser; and putting the dispensed membrane in a 45° C. drying oven for drying for 1 h.

2) Preparation of a fluorescent microsphere-labelled monoclonal antibody against bovine serum amyloid A:

a) diluting 1 mg of fluorescent microspheres to 1 mL with 50 mM IVIES buffer (pH 6.0);

b) weighing about 2 mg of Sulfo-NHS and about 2 mg of EDC, diluting the Sulfo-NHS to 10 mg/ml with 50 mM MES buffer (pH 6.0), adding 0.5 mg of the diluted Sulfo-NHS to diluted fluorescent microspheres, and mixing well; diluting the EDC to 10 mg/ml with 50 mM MES buffer (pH 6.0), adding 0.5 mg of the diluted EDC to the diluted fluorescent microspheres, and mixing well at room temperature for reaction for 30 min;

c) centrifuging at 12000 rpm at 4° C. for 20 min, removing the supernatant, and adding 1 mL of 50 mM boric acid buffer (pH 8.0) for resuspension;

d) to 100 μg of monoclonal antibody against bovine serum amyloid A (purchased from Medix Biochemica), adding fluorescent microspheres, and mixing well at room temperature for reaction for 1 h;

e) adding 50 ul of 5% BSA blocking solution, and mixing well at room temperature for reaction for 30 min; and

f) centrifuging at 12000 rpm at 4° C. for 20 min, removing the supernatant, and adding 1 mL microsphere preservation solution (containing 0.5% BSA and 20% sucrose) for resuspension.

3) Preparation of fluorescent microsphere-labelled chicken IgY:

a) diluting 1 mg of fluorescent microspheres to 1 mL with 50 mM IVIES buffer (pH 6.0);

b) weighing about 2 mg of Sulfo-NHS and about 2 mg of EDC, diluting the Sulfo-NHS to 10 mg/ml with 50 mM MES buffer (pH 6.0), adding 0.25 mg of the diluted Sulfo-NHS to diluted fluorescent microspheres, and mixing well; diluting the EDC to 10 mg/ml with 50 mM MES buffer (pH 6.0), adding 0.25 mg of the diluted EDC to the diluted fluorescent microspheres, and mixing well at room temperature for reaction for 30 min;

c) centrifuging at 12000 rpm at 4° C. for 20 min, removing the supernatant, and adding 1 mL of 50 mM boric acid buffer (pH 8.0) for resuspension;

d) to 15 μg of chicken IgY (purchased from Nanjing Hanrui Baike Biotechnology Co., Ltd.), adding fluorescent microspheres, and mixing well at room temperature for reaction for 1 h;

e) adding 50 ul of 5% BSA blocking solution, and mixing well at room temperature for reaction for 30 min; and

f) centrifuging at 12000 rpm at 4° C. for 20 min, removing the supernatant, and adding 1 mL microsphere preservation solution (containing 0.5% BSA and 20% sucrose) for resuspension.

4) Preparation of a binding pad

a) preparing a microsphere dispensing working solution at a ratio of 5:1:6 of T line fluorescent microsphere-labelled antibody:C line fluorescent microsphere-labelled antibody:fluorescent microsphere-labelled antibody preservation solution;

b) cutting a pretreated binding pad into 10 mm wide strips, and dispensing the microsphere dispensing working solution on the binding pad at 8 μl/cm by using a XYZ platform dispenser; and

c) and putting the dispensed membrane in a 45° C. drying oven for drying for 1 h.

5) Cutting a sample pad: cutting the pretreated sample pad into 22 mm wide strips.

6) Cutting an absorbent paper: cutting the absorbent paper into 31 mm wide strips.

7) Assembling a test reagent card: lapping and pasting one end of the nitrocellulose membrane close to the C line on the absorbent paper, with the other end of the nitrocellulose membrane close to the T line lapped and adhered on the binding pad, and finally lapping and pasting the sample pad beside the binding pad, and cutting the adhered test paper board into 80 mm long and 4 mm wide test paper strips.

8) Assembling a case: putting the test reagent card into the matched plastic case, and compressing the upper and lower covers.

9) Preparation of a sample diluent: the sample diluent is prepared from 0.01M PBS (pH 7.4), containing 0.5% Tween-20 and 0.1% casein.

### Embodiment 2: Plotting a Calibration Curve of a Fluorescence Immunochromatographic Assay Kit for Bovine Serum Amyloid A

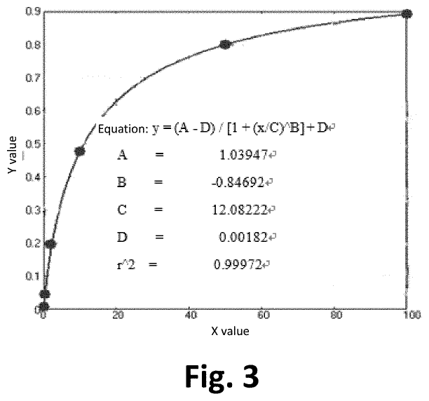

Using 250 mg/L sample determined by a bovine serum amyloid ELISA kit developed by Shanghai BlueGene Biotech Co., Ltd. as a high-value reference sample, and diluting the sample with the sample diluent in the kit of the present invention to obtain calibrator concentration points of 0 mg/L, 0.4 mg/L, 2 mg/L, 10 mg/L, 50 mg/L and 100 mg/L; Testing each calibrator concentration point for 10 times by using the test reagent card in the kit of the present invention, and performing a four-parameter fit with the T/C mean value measured at each calibrator concentration point to the corresponding theoretical concentration value to plot a calibration curve (FIG. 3), where the X-axis is the calibrator concentration point and the Y-axis is the T/C mean value.

### Embodiment 3: Limit of Blank Test by Fluorescence Immunochromatographic Assay Kit for Bovine Serum Amyloid A

According to relevant literatures, the sensitivity of the kit was evaluated by testing the limit of blank.

Testing the 0 mg/L calibrator concentration point for 20 times by using the test reagent card in the kit of the present invention, calculating the T/C mean value and standard deviation at this point, and substituting the (mean value+2X standard deviation) into a calibration curve equation to calculate a limit of blank of 0.052 mg/L, which indicates that the test result will be >0 mg/L for samples with content of serum amyloid A in milk >0.052 mg/L as determined by the kit of the present invention.

According to some literatures, the reference content of serum amyloid A in milk samples is <0.55 mg/L. The value indicates that the content of serum amyloid A in milk samples of healthy dairy cows is usually <0.55 mg/L, while dairy cows with the content of serum amyloid A in samples ≥0.55 mg/L may suffer from mastitis or other diseases.

If samples with concentration ≥0.55 mg/L are tested by the kit developed in the present invention, it will be possible to obtain results of >0 mg/L, and the content of serum amyloid A can be calculated based on the calibration curve, so as to help judge the health status of dairy cows. Therefore, the sensitivity of the kit developed by the method can meet the detection requirements.

TABLE 1
Limit of blank

| No. | T/C |
|---|---|
| 1 | 0.007 |
| 2 | 0.007 |
| 3 | 0.005 |
| 4 | 0.005 |
| 5 | 0.010 |
| 6 | 0.007 |
| 7 | 0.005 |
| 8 | 0.006 |
| 9 | 0.010 |
| 10 | 0.010 |
| 11 | 0.008 |
| 12 | 0.010 |
| 13 | 0.005 |
| 14 | 0.004 |
| 15 | 0.007 |
| 16 | 0.007 |
| 17 | 0.008 |
| 18 | 0.004 |
| 19 | 0.003 |
| 20 | 0.011 |
| Mean value | 0.007 |
| Mean value + | 0.012 |
| 2X standard | |
| deviation | |
| Standard | 0.0024 |
| deviation | |
| Limit of blank | 0.052 |

The above description is only embodiments of the present invention, and thus does not limit the scope of the patent of the present invention. Any equivalent structure or equivalent process transformation made by using the specification of the present invention and the contents of the drawings, or directly or indirectly applied to other related technical fields are equally included in the scope of patent protection of the present invention.

1. A kit for detecting mastitis in dairy cows, comprising a plastic case, a test reagent card and a sample diluent, wherein the case comprises a bottom case and an upper cover, wherein a test strip slot is formed in the bottom case, and a scan window and a sample loading hole are arranged on the upper cover; characterized in that the test reagent card consists of a sample pad, a binding pad, a nitrocellulose membrane and an absorbent paper which are sequentially adhered on a bottom board; the position of the scan window is matched with the position of the nitrocellulose membrane, and the position of the sample loading hole is matched with the position of the sample pad.

2. The kit for detecting mastitis in dairy cows according to claim 1, characterized in that both the sample pad and the binding pad are glass cellulose membranes, and the bottom board is a PVC bottom board.

3. The kit for detecting mastitis in dairy cows according to claim 2, characterized in that the sample pad is pretreated with a sample pad pretreatment buffer which is prepared by dissolving a sample pad buffer, a sample pad protein protectant and a sample pad surfactant in water; wherein, the sample pad buffer is selected from any one of PBS buffer, Tris-HCl buffer, borate buffer and citric acid-sodium citrate buffer, with a concentration of 5-100 mM; the sample pad protein protectant is selected from any one or more of BSA, gelatin from cold water fish skin, casein, casein sodium salt and bovine serum, with a concentration of 0.5-20 g/L; the sample pad surfactant is selected from any one of Tween-20, Tween-80, TritonX-100 and TritonX-305, with a concentration of 2-20 g/L; and pH value of the sample pad pretreatment buffer is adjusted by using a pH regulator commonly used in the prior art, with a range of 7.0-8.0.

4. The kit for detecting mastitis in dairy cows according to claim 2, characterized in that the binding pad contains a complex of fluorescent microsphere-labelled chicken IgY and a fluorescent microsphere-labelled monoclonal antibody against bovine serum amyloid A; wherein the binding pad is pretreated by using a binding pad pretreatment buffer which is prepared by dissolving a binding pad protein protectant, a binding pad reaction enhancer and a binding pad surfactant in water; wherein, the binding pad protein protectant is selected from any one or more of bovine serum albumin (BSA), gelatin from cold water fish skin, casein, casein sodium salt, bovine serum, sucrose and trehalose, with a concentration of 0.5-50 g/L; the binding pad reaction enhancer is selected from any one of PEG6000, PEG8000, PEG20000, PVP K30 and PVP K40, with a concentration of 0.1-10 g/L; and the binding pad surfactant is selected from any one of Tween-20, Tween-80, TritonX-100 and TritonX-305, with a concentration of 0.5-10 g/L.

5. The kit for detecting mastitis in dairy cows according to claim 2, characterized in that the nitrocellulose membrane is coated with a test line of a monoclonal antibody against bovine serum amyloid A and a quality control line of a rabbit anti-chicken IgY antibody, wherein the test line is close to the binding pad and the quality control line is close to the absorbent paper.

6. The kit for detecting mastitis in dairy cows according to claim 3, characterized in that the binding pad and the sample pad are pretreated in the following steps: soaking the binding pad or the sample pad in the binding pad pretreatment buffer or the sample pad pretreatment buffer respectively for 0.5-2 h, then taking out and drying the binding pad or the sample pad at 36-38° C.

7. An application method of the kit for detecting mastitis in dairy cows according to claim 1, characterized by comprising the following steps:
dripping the sample into the sample loading hole to allow the sample to flow into the binding pad by chromatography, wherein the sample, if containing bovine serum amyloid A, binds to the fluorescent labelled monoclonal antibody against bovine serum amyloid A on the binding pad to form an immune complex, the complex and the fluorescent labelled chicken IgY continue to move to the nitrocellulose membrane where the complex specifically binds to a T line coated monoclonal antibody against bovine serum amyloid A, finally forming a double antibody sandwich complex, and the fluorescent labelled chicken IgY binds to a C line coated rabbit anti-chicken IgY antibody;
measuring and analyzing fluorescence values of the T line and the C line by using a quantitative fluorescence analyzer; plotting a calibration curve based on the relationship between the fluorescence ratio of T/C measured by the kit and the concentration of a calibrator, substituting the measured fluorescence ratio of T/C into the calibration curve, and calculating the content of bovine serum amyloid A in the sample.

8. The kit for detecting mastitis in dairy cows according to claim 4, characterized in that the binding pad and the sample pad are pretreated in the following steps: soaking the binding pad or the sample pad in the binding pad pretreatment buffer or the sample pad pretreatment buffer respectively for 0.5-2 h, then taking out and drying the binding pad or the sample pad at 36-38° C.
